(19) United States
(12) Patent Application Publication
(10) Pub. No.: US 2007/0248642 A1
(43) Pub. Date: Oct. 25, 2007

(54) Foam and use thereof

(76) Inventors: Michael Dornish, Bekkestua (NO); Therese Andersen, Sande I Vestfold (NO); Jan Egil Melvik, Oslo (NO)

Correspondence Address: Patent Administrator;FMC Corporation, 1735 Market Street, Philadelphia, PA, 19103, US

(21) Appl. No.: 11/712,789

(22) Filed: Mar. 1, 2007

Related U.S. Application Data
(60) Provisional application No. 60/793,301, filed on Apr. 19, 2006.
(60) Provisional application No. 60/794,619, filed on Apr. 24, 2006.

(51) Int. Cl. A61K 9/70; A61K 47/38; A61K 47/46

(52) U.S. Cl. 424/423; 424/443; 514/779; 514/781

(57) ABSTRACT

The invention provides a method of producing a gelled foam comprising the steps of:

forming a dispersion by mixing i) a solution comprising a soluble polysaccharide and a plasticizer and adding a polysaccharide/gel-forming ion particles or ii) a soluble, preferably immediately soluble, polysaccharide, preferably an alginate, a polysaccharide/gel-forming ion particles, and adding a solvent, said dispersion (ii) further comprising a water soluble plasticizer to make the dispersion and then aerating the dispersion to form the foam. The foam may be inhomogeneous in structure which is useful in providing improved delivery of a component carried in the foam and in degradation.

This application claims the benefit of U.S. Provisional Application No. 60/793,301, filed Apr. 19, 2006, and U.S. Provisional Application No. 60/794,619, filed Apr. 24, 2006.

The invention relates to a foam formed from a dispersed polysaccharide/gel-forming ion particulates, particularly to a gelled foam formed from a soluble alginate gelled by ions. The invention also relates to a device and a structure containing such a foam for example a composite of a foam and a polysaccharide gel, a method for making the device or structure, and use of the device or structure.

Alginate systems which have a delayed gelling process and a compositions comprising immediately soluble alginate and alginate/gel-forming ion particles for preparing alginate gels and devices, kits and methods of making and using such systems are disclosed in U.S. Patent Application No. 11/248,984 (Melvik) “Self Gelling Alginate Systems and Uses thereof”.

U.S. Pat. No. 6,656,974 B1 (Renn) discloses methods of producing integral absorbent alginate foam materials for wound care dressings where calcium/sodium alginate fibres or other calcium/sodium particulate materials preferably having 60 to 85% in the calcium salt form donate calcium ions to crosslink the alginate polymer in the precursor foam.

Gelled biopolymer foams and a method for manufacture are disclosed in WO05023323 (Gaserod) in which the gelling is initiated by release of gel-forming ions from a gelling agent responsive to pH change from a pH modifier. An acidic environment is created when a pH modifier such as D-glucono-δ-lactone (GDL) is used during the gelling process.

The present invention relates to a method of producing a gelled foam, preferably to a self-gelling alginate foam, comprising the steps of:
(a) forming a dispersion by mixing i) a solution comprising a soluble polysaccharide and a plasticizer and adding a polysaccharide/gel-forming ion particles or ii) a soluble, preferably immediately soluble, polysaccharide, preferably an alginate, a polysaccharide/gel-forming ion particles, and adding a solvent, said dispersion (ii) further comprising a water soluble plasticizer;
(b) aerating the dispersion in (a), said dispersion optionally further comprising a foaming agent;
(c) optionally dispensing the wet foam; and
(d) optionally drying the wet foam.

In a second aspect, the invention provides a foam produced according to the method of the invention. In a third aspect the invention provides a composite comprising a foam of the present invention and a polysaccharide which has been formed into a gel by interaction with the gel-forming ions in the foam. In a fourth aspect the invention provides a method of using the foam and uses of the foam.

Foams produced according to the method of the invention may have the soluble polysaccharide from the solution and the polysaccharide of the particle non-uniformly distributed through the foam. Preferably the structure of the foam is inhomogeneous. This provides advantage due to disconformity or discontinuity of the structure of the foam which may enable leaching of materials or components from the foam so providing improved degradation and delivery of components from the foam.

The present invention further relates to a method of producing a device and a structure comprising a self-gelling foam. The method comprises forming a foam from a self gelling polysaccharide dispersion comprising a soluble polysaccharide, preferably an alginate, a plasticizer, a solvent and fine dispersible polysaccharide/gel-forming ion particles, and optionally dispensing the wet foam. Optionally, the foam may be shaped with or without addition of films, fibres, meshs, or other structural elements. Suitably, the foam is dried. In one embodiment, the structure comprises one or more self-gelling formulations which may be added sequentially or simultaneously as a self-gelling foam or as a solution and optionally the foam is dried.

The present invention further relates to a method of forming a polysaccharide, preferably alginate foam comprising biomaterials for example tissue or cells and uses thereof. Such tissue or cells may be dosed, for example from saline, directly to the foam or dosed as a dispersion of cells or tissue in a polysaccharide solution into the foam.

The present invention further relates to a method for using a self-gelling polysaccharide foam as a cell culture matrix, tissue engineering scaffold, a topical wound healing bandage, an anti-adhesion barrier, or as a delivery device for pharmaceutical, cells or actives.

A foam according to the invention is suitably prepared by mixing a soluble polysaccharide; and polysaccharide/gel-forming ion particles in the presence of a plasticizer and a solvent to form a dispersion and aerating the dispersion. The self-gelling process is initiated by mixing the soluble polysaccharide with the polysaccharide/gel-forming ion particles suitably by agitation for example, by stirring or by using a suitable mixing device. Suitably air may be incorporated during mixing so as to dispense the polysaccharide/gel-forming ion particles within the soluble polysaccharide solution.

A foaming agent may be used to increase the amount of air which can be incorporated into the foam and/or to retard the rate of foam collapse. Suitable foaming agents include ionic or non ionic surfactants, for example Tween 20, albumin or foam stabilizing hydrocolloids or combinations thereof for example as disclosed in WO05023323 or U.S. Pat. No. 6,656,974 which are incorporated by reference. In some embodiments, it is preferred to use foaming agents which are foam stabilizing hydrocolloids for example hydroxylpropylmethylcellulose (HPMC) and methyl cellulose and albumin. In an especially preferred embodiment, the foaming agent is polymeric and desirably biologically acceptable. For applications in or on the human or animal body, the foaming agent is preferably substantially free of a non-polymeric surfactant.

A foaming agent is preferably added prior to the incorporation of the polysaccharide/gel-forming ion particles. The type and level required are dependent upon the desired foam density and manufacturing process The foam density will be dependent upon a number of factors including the amount of incorporated air, drying temperature, amount of polysaccharide/gel-forming ion particles, particle size of the polysaccharide/gel-forming ion particles and the amount of foaming agent and molecular weight and concentration of the polysaccharide.

Additional ingredients may be incorporated if desired to modify the foam properties, for example texture, absorbency, color, strength, and the like or to provide specific functionality for example by providing delivery of a pharmaceutical or in carrying cells, so long as the resulting foam is suited for the desired application.

Without wishing to be bound by any theory it is believed that the wet foam begins to gel as the gel-forming ion from the polysaccharide/gel-forming ion particles begins cross linking polysaccharide polymers from the polysaccharide/gel-forming ion particles and the soluble polysaccharide polymers in solution. Furthermore it is believed that the gelling kinetics of the formulation are dependent upon several factors including: the concentration of the soluble alginate in solution, the concentration of the polysaccharide particles in the dispersion, the relative content of gel-forming ion to polysaccharide, the presence of non-gel-forming ions or other polymers or carbohydrates, temperature, the size of polysaccharide/gel-forming ion particles, the presence of impurities, and the types of polysaccharide used, as well as the manufacturing process for the polysaccharide particles and post manufacturing treatment of polysaccharide starting materials. This polysaccharide system may therefore be adapted to each particular application.

Self gelling formulations suitable for use as foams may be used to prepare biostructures in combination with formulation suitable for use as gels. For biostructures which include the support or entrapment of cells, multicellular aggregates, tissues or other biomaterials within the forming gel, the solvent, the polysaccharide solution or the dispersion may be premixed with the material to be supported or entrapped.

The foam may be dispensed and optionally shaped prior to drying, for example onto a substrate, into mold, extruded and cut, portioned into an air stream, or applied to or within an individual at a site where the foam is desired. Polysaccharide gel formation, initiated when the soluble polysaccharide and polysaccharide/gel-forming ion particles are mixed in the presence of a solvent, continues and the polysaccharide foam is gelled, for example it sets in situ.

As used herein, the term “self-gelling” as employed herein refers to the gelling process which occurs when the soluble polysaccharide and polysaccharide/gel-forming ion particles are mixed in the presence of a solvent. A “self gelling polysaccharide” is an polysaccharide dispersion which includes soluble polysaccharide and polysaccharide/gel-forming ion particles in a solvent or is an polysaccharide gel which is formed from a soluble polysaccharide and polysaccharide/gel-forming ion particles in a solvent.

The components used in producing the self-gelling polysaccharide may be maintained prior to use in any of several forms. For example, the soluble polysaccharide may be maintained in solution or as a powder. In some embodiments, the soluble polysaccharide may be maintained as a powder that is immediately soluble such as when it is freeze dried. Similarly, the polysaccharide/gel-forming ion particles may be maintained as a dispersion or as a powder.

The polysaccharide polymers or combinations thereof used in the soluble polysaccharide may be the same or different from those in the polysaccharide/gel-forming ion particles.

The concentration of polysaccharide, both soluble polysaccharide and the polysaccharide in the particles in a dispersion relative to the amount of solvent affects gelling time, porosity, stability and biodegradability, gel strength and elasticity of the gel. Gelled foam having specific properties may be prepared by using specific ratios of soluble polysaccharide and polysaccharide/gel-forming ion particles to solvent. Generally, the lower the concentration of polysaccharide (for a given ratio of soluble polysaccharide to polysaccharide), the more biodegradable a gel will be. Suitably, the level of polysaccharide is at least 1%, more preferably at least 5% and may be more than 10% by weight In some embodiments, 0.5%, 0.75%, 1%, 1.25%, 1.5%, 2%, 2.5%, 3%, 4%, 5%, 6%, 7%, 8%, 9%, 10% of the or more alginate (soluble polysaccharide and polysaccharide in the form of polysaccharide/gel-forming ion particles) may be used.

The relative concentration of the soluble polysaccharide to polysaccharide in the form of polysaccharide/gel-forming ion particles in the dispersion affects gelling time, pore size, tensile strength and elasticity of the foams as well as stability and biodegradability. Foams having specific properties may be prepared by using specific ratios of soluble polysaccharide to polysaccharide/gel-forming ion particles.

In a preferred embodiment, the ratio of the concentration of soluble polysaccharide o the concentration of polysaccharide in the form of polysaccharide/gel-forming ion particles is from 10:1 to 1 to 10 and preferably from 7 to 1:1 to 2. Generally, the less gel-forming ion present, the more biodegradable a gel will be. Reducing the concentration of polysaccharide/gel-forming ion in the system may be used to create gels with lower stability and higher biodegradability as the gel network is less saturated with cross-linking ions. Self gelling allows for the preparation of foams with lower concentrations of gel-forming ion to produce gels particularly well suited for biodegradable uses.

Where the polysaccharide comprises alginate, the relative content of G and M monomers in the alginate polymers affects pore size of the gel, stability and biodegradability, gel strength and elasticity of the gels (i.e. the alginate gel matrix of the foam). Alginate polymers contains large variations in the total content of M and G, and the relative content of sequence structures also varies largely (G-blocks, M-blocks and MG alternating sequences) as well as the length of the sequences along the polymer chain. Generally, the lower the G content relative to M content in the alginate polymers used the more biodegradable a gel will be. Gels with high G content alginate generally have larger pore sizes and stronger gel strength relative to gels with high M alginate, which have smaller gel pore sizes and lower gel strength.

In a preferred embodiment, one or more of the alginate polymers of the alginate foam contain more than 50% α-L-guluronic acid. In some embodiments, one or more of the alginate polymers of the alginate foam contain more than 60% α-L-guluronic acid and preferably 60% to 80% α-L-guluronic acid, especially 65% to 75% α-L-guluronic acid. In some embodiments, one or more of the alginate polymers of the alginate foam contain more than 70% α-L-guluronic acid. In some embodiments, one or more of the alginate polymers of the alginate foam contain more than 50%, preferably more than 60% C-5 epimer β-D-mannuronic acid and especially 60% to 80%, for example 65% to 75% C-5 epimer β-D-mannuronic acid. In some embodiments, one or more of the alginate polymers of the alginate foam contain more than 70% C-5 epimer β-D-mannuronic acid. Procedures for producing uronic blocks from are disclosed in U.S. Pat. No. 6,121,441. G-block alginate polymers and their uses as modulators of alginate gel properties are disclosed in U.S. Pat. No. 6,407,226. Preferably the G-block content of an alginate polymer is at least 30%, preferably at least 50%, and may be more than 60 or more than 70%. Some preferred embodiments include 30% G, 35% G, 40% G, 45% G, 50% G, 55% G, 60% G, 65% G, 70% G, 75%, 80% G or 85% G.

A polysaccharide polymer may have an average molecular weights ranging from 2 to 1000 kD or from 50 to 500 kD. In some embodiments, the polysaccharide polymer of the foam has an average molecule weight of from 2 to 350 kD or 3 to 350 kD. In some embodiments, the polysaccharide polymer of the foam has an average molecule weight of from 2 to 100 kD. In some embodiments, gels are designed to have a high degree of biodegradability and suitably have a lower level of polysaccharide, less gel-forming ion, and where the polysaccharide is an alginate, lower G content and lower molecular weight alginates can be produced using the lower limits of one or more of these parameters as set forth herein to produce gels with a high degree of biodegradability.

The polysaccharide may possess a viscosity in a 1% solution measured at 20 degrees centigrade of from 25 to 1000 mPas and in some embodiments, preferentially 50 to 1000 mPas (1% solution, 20 degrees C.).

In some embodiments, the viscosity of the soluble polysaccharide is lower to improve biodegradability, preferably less than 550 mPa-s, more preferably less than 500 mPa-s, or it may be less than 450 mPa-s, less than 400 mPa-s, or even less than 350 mPa-s (1% solution, 20 degrees C.).

In some embodiments, it is preferred that methods of manufacture of polysaccharide/gel-forming ion particles provide products with a controlled stoichiometric amount of gel-forming ion. The polysaccharide/gel-forming ion particles may provide products with stoichiometric (100% saturation) amount of said gel-forming ions or the level may be sub-stoichiometric amount (<100% saturation) of said gel-forming ion. Use of salts with controlled stoichiometry imparts greater reproducibility in the self-gelling polysaccharide systems. Use of such sub-stoichiometric salts imparts improved biodegradability to self-gelling polysaccharide foams.

Examples of polysaccharides suitable for use in the polysaccharide/gel-forming ion particle include alginates, pectins, carrageenans, hyaluronates, chitosan and mixtures thereof. These polysaccharides are also suitable for use in the soluble polysaccharide provided that the aqeous dispersion is able to form a wet foam. The foam may be prepared using a single polysaccharide or alternatively from more than one polysaccharide. The polysaccharide in the solution and the particle may be then same or different. Alginates, chitosan and hyaluronates are preferred polysaccharides.

Suitable polysaccharides for use in the present invention include those that are soluble in a solvent, such as water, and can be formed into a gel by interaction with gel-forming ions. Examples of suitable polysaccharides include alginates, pectins, carrageenans, chitosan, hyaluronates, and mixtures thereof provided that the polysaccharide alone or in a mixture with another polysaccharide may form a gel. Alginates are a preferred polysaccharide for use in the present invention.

In a preferred embodiment, the polysaccharide comprises an ultrapure polysaccharide possessing a low content of endotoxins for example less than 350 EU/g, preferably less than 100 EU/g. either for the particle or as the soluble polysaccharide, or both, as appropriate. For example, when alginates are used for implantation within the human body, the alginates suitably have an endotoxin content of less than 100 EU/g. In a preferred embodiment the composite has an endotoxin content of less than 10 EU/g

In some embodiments, the alginate has an endotoxin level of less than 500 EU/gram, less than 450 EU/gram, less than 400 EU/gram, less than 350 EU/gram, less than 300 EU/gram, less than 250 EU/gram, less than 200 EU/gram, less than 150 EU/gram, less than 100 EU/gram, less than 75 EU/gram less than 50 EU/gram or less than 25 EU/gram.

Ultrapure alginate is commercially available such as from different sources of seaweed like Laminaria Hyperborea. Commercial calcium salts of alginic acid are generally manufactured in processes whereby calcium is added to alginic acid in the solid phase by simple admixture and kneading of the components together. Examples of commercially available calcium salts of alginic acid are Protaweld (from FMC BioPolymer) and Kelset from ISP Corporation. The alginate/gel-forming ion particles may be produced using ultrapure alginate by making an alginate gel using the ultrapure alginate and a gel-forming ion, washing out sodium or other ions that were present in the ultrapure alginate, drying the gel to remove the water, and making particles from the dried gel. In some embodiments, the alginate/gel-forming ion particles are stoichiometric salts. Alginate/gel-forming ion particles preferably have a high purity and a specific, consistent and generally uniform content of gel-forming ion such as, for example, calcium or strontium barium, zinc, iron, manganese, copper, lead, cobalt, nickel, or combinations thereof, such that gel formation speed and gel strength can be provided with more precise predictability. Insoluble alkaline earth salts of alginic acid such as for example calcium alginate or strontium alginate (depending upon the gel-forming ion used) or insoluble transition metal salts of alginic acid (such as those using gel-forming ions of copper, nickel, zinc, lead, iron, manganese or cobalt) can be manufactured with a known and predetermined content of alkaline earth ions by precipitation from the solutions. In some embodiments, commercially available sodium alginate is first used to prepare a sodium alginate solution. Optionally, sodium salt such as sodium carbonate may be included in the sodium alginate solution. A salt containing the desired gel-forming ion for the alginate/gel-forming ion particle, such as for example, calcium salt or strontium salt such as calcium chloride or strontium chloride, is used to make a solution. The sodium alginate solution is combined, preferably slowly, with the gel-forming ion solution. Preferably, the combined solutions are continuously stirred during the mixing process. Alginate such as for example calcium alginate or strontium alginate (depending upon the gel-forming ion used) precipitates from the combined solutions. The precipitated alginate is then be removed from the solution and washed repeatedly, such as 2-10 times, with purified water for example to remove all soluble ions. The removal of soluble ions is confirmed for example by testing the conductivity of alginate in purified water compared to the conductivity of purified water. After washing, the alginate can be dried, such as with a vacuum. The dried alginate can be milled and, in some embodiments, selected for particle sizes.

In some embodiments, the polysaccharide may be sterilized, preferably by γ-irradiation, E-beam, ethylene oxide, autoclaving or contacting the foam with alcohol prior to addition of the liquid component or contacting with NOx gases, hydrogen gas plasma sterilization. Sterilisation should not be employed where it adversely affects the foam, or a functional component contained in the foam.

In some preferred embodiments, the polysaccharide is sterile ultrapure polysaccharide, for example sterile ultrapure alginate. Conditions often used to sterilize material can change the polysaccharide, such as decrease the molecular weight. In some embodiments, the sterile polysaccharide may be produced using sterility filters.

In some embodiments, the polysaccharide foam is may be coated, e.g. with a polycationic polymer like a poly amino acid or chitosan after the gel matrix forms. In some embodiments, poly-lysine is the polycationic polymer. In some embodiments, poly-lysine is linked to another moiety and the poly-lysine is thus used to facilitate association of the moiety to the gel. Examples of moieties linked to the gel using polycationic polymers include, for example, drugs, peptides, contrast reagents, receptor binding ligands or other detectable labels. Some specific examples include vascular endothelial growth factor (VEGF), epidermal growth factor (EGF), transforming growth factor (TGF), and bone morphogenic protein (BMP). Drugs may include cancer chemotherapeutic agents such as Taxol, cis-platin and/or other platinum-containing derivatives. Carbohydrate polymers may include hyaluronan, chitosan, heparin, laminarin, fucoidan, chondroitin sulfate. In some embodiments, the alginates used are modified alginate polymers such as chemically modified alginate in which one or more polymers are linked to a different alginate polymer. Examples of such modified alginate polymers may be found in U.S. Pat. No. 6,642,363, which is incorporated herein by reference.

In some embodiments, the polysaccharide polymer may include a functional component such as, for example, a pharmaceutical, a population of cells, a peptide, a contrast reagent, a receptor binding ligand or other detectable label. In one embodiment, the polysaccharide polymer includes an RDG peptide (Arg-Asp-Gly), a radioactive moiety (e.g. ¹³¹I) or a radio opaque substance. Other examples of moieties linked to polysaccharide polymer includes, for example, pharmaceutical, a peptide, contrast reagents, receptor binding ligands or other detectable labels. Some specific examples include vascular endothelial growth factor (VEGF), epidermal growth factor (EGF), transforming growth factor (TGF), and bone morphogenic protein (BMP). Pharmaceuticals may include cancer chemotherapeutic agents such as Taxol, cis-platin and/or other platinum-containing derivatives. Carbohydrate polymers may include hyaluronan, chitosan, heparin, laminarin, fucoidan, chondroitin sulfate.

The soluble polysaccharide may be a salt such as, for example, a sodium, potassium or ammonium salt, for example Na⁺-alginate, K⁺-alginate, NH₄-alginate or combinations thereof. In some embodiments, the soluble polysaccharide may be freeze dried or otherwise desiccated. Freeze dried soluble polysaccharides may be “immediately soluble.” “Immediately soluble” means in this context that the material is soluble in water in less than one minute, preferably less than 30 seconds, more preferably less than 15 seconds. “Readily soluble” materials in this context take more than one minute and usually several minutes to go into solution.

The gel-forming ions used in the alginate/gel-forming ion particles affects gelling kinetics, gel strength, and elasticity. Gel-forming ions also have affects on cell growth. The gel-forming ions used in the alginate/gel-forming ion particles may be Ca⁺⁺, Sr⁺⁺, Ba⁺⁺, Zn⁺⁺, Fe⁺⁺, Mn⁺⁺, Cu⁺⁺, Pb, Co, Ni, or combinations thereof. Preferred gel-forming ions used in alginate/gel-forming ion particles are Ca⁺⁺, Sr⁺⁺, and Ba⁺⁺. More referred gel-forming ions used in alginate/gel-forming ion particles are Ca⁺⁺, and Sr⁺⁺.

The polysaccharide gel-forming ion complexes are particles. The particles are generally non fibrous based on a L/D ratio where the particle shape is characterized by a largest dimension (L) and smallest dimension (D). Non-fibrous L/D is less than 10, preferably less than 5, preferably less than 2. An L/D of 10 or more is a chopped fiber. The polysaccharide/gel-forming ion can be maintained as a dispersion or in dry form. If the former, the dispersion can be mixed with a solution containing soluble polysaccharide or with immediately soluble polysaccharide to form a dispersion of polysaccharide/gel-forming ion particles in a solution containing soluble alginate. If the polysaccharide gel-forming ion particles are in dry form, they may be mixed with dry immediately soluble alginate and subsequently with a solution to form a dispersion of polysaccharide/gel-forming ion particles in a solution containing soluble polysaccharide or the dry polysaccharide gel-forming ion particles may be combined with a solution containing soluble polysaccharide to form a dispersion of polysaccharide gel-forming ion particles in a solution containing soluble polysaccharide.

Suitably, the agitation that occurs upon mixing the components to form the dispersion results in distribution of the solid particles within the solution. The dispersion so produced can be in the form of a slurry which is foamed e.g. by physical means (whipping pressure differential, gas injection or extrusion ), and then dispensed, e.g. extruded or cast onto a substrate to self gel, or poured or injected to self gel within a mold or cavity to form the shape of such mold or cavity.

Suitably, the wet foam of polysaccharide/gel-forming ion particles in a solution containing soluble polysaccharide is formed, it is dispensed to the site where the self gelling occurs to form a gelled polysaccharide foam. In some embodiments, the dispersion may be dispensed to a site in vivo. In some embodiments, the foamed dispersion is dispensed on to a site on a mammalian body. In some embodiments, the foamed dispersion is dispensed into a mold or other container or surface.

The concentration of gel-forming ions used in the polysaccharide/gel-forming ion particle affects gelling kinetics, gel strength, and elasticity. The higher the concentration of gel-forming ions, the higher the strength. Strength is highest when the polysaccharide is saturated with gel-forming ion. Conversely, the lower the concentration of gel-forming ion, the lower the strength and higher the degree of biodegradability.

The foam structures of the invention can be made to immediately disintegrate upon hydration or it can be made with a more structural integrity. For example for an alginate foam, foam characteristics and degradation are dependent upon several factors: 1) The ratio between the soluble alginate and the Ca— or Sr-alginate; 2) The content/saturation of divalent cations of the Ca— or Sr-alginate; 3) The monomeric content of the alginate (G-rich or M-rich); and 4) the particle size of the Ca— or Sr-alginate. The self-supporting foams can be dissolved by adding a sequestering agent for the gel-forming ions e.g. an aqueous solution of citrate, EDTA or hexametaphosphate.

Suitably, the polysaccharide/gel-forming ion particle has a particle size of about 500 microns to about 0.001 microns, more preferably from about 100 microns to about 0.01 microns, even more preferably from about 50 microns to about 0.1 microns. In some embodiments, the particles may be fractionated.

The particle size of the polysaccharide/gel-forming ion particles may affect the gelling kinetics and the final properties of the gel. The smaller the particle size the more rapid the completion of gel formation. Larger particle sizes produce stronger gels. Particle sizes may be controlled by, for example, sifting polysaccharide/gel-forming ion particles through various different size filters such that the particles can be generally all be within a predetermined size range. In some embodiments, particles are <25 μm, 25-45 μm, 45-75 μm, 75-125 μm or >125 μm.

The solvent used may be, for example, water, saline, sugar solution, cell culture solution, a solution such as a pharmaceutical solution, protein, or nucleic acid solution, a suspension such as a cell suspension, liposomes, or a contrast reagent suspension.

The polysaccharide gelled foam formed may comprise, for example, a pharmaceutical, nucleic acid molecules, cells, multicellular aggregates, tissue, proteins, enzymes, liposomes, a contrast reagent or a biologically active material. Examples of a biologically active material are hyaluronate and chitosan. Contrast reagents include tantalum and gadolinium. Some specific examples of proteins include vascular endothelial growth factor (VEGF), epidermal growth factor (EGF), transforming growth factor (TGF), and bone morphogenic protein (BMP). Drugs may include cancer chemotherapeutic agents such as Taxol, cis-platin and/or other platinum-containing derivatives. Carbohydrate polymers may include hyaluronan, chitosan, heparin, laminarin, fucoidan, chondroitin sulfate.

The cells that can be used in the foams include non-recombinant and recombinant cells. In some embodiments cells are added to the foam directly or alternatively the cells are dispersed in an alginate solution to encapsulate the cells. In some embodiments the cells are mammalian cells, preferably human cells. In some embodiments in which encapsulated cells are non-proliferating cells, the non-proliferating cells may be selected from the group consisting of: islets of Langerhan, hepatic cells, neural cells, renal cortex cells, vascular endothelial cells, thyroid and parathyroid cells, adrenal cells, thymic cells, ovarian cells and chondrocytes. In some embodiments in which encapsulated cells are proliferating cells, the proliferating cells may be stem cells, progenitor cells, proliferating cells of specific organs, fibroblasts and keratinocytes or cells derived from established cell lines, such as for example, 293, MDCK and C2C12 cell lines. In some embodiments, encapsulated cells comprise an expression vector that encodes one or more proteins that are expressed when the cells are maintained. In some embodiments, the protein is a cytokine, a growth factor, insulin or an angiogenesis inhibitor such as angiostatin or endostatin, other therapeutic proteins or other therapeutic molecules such as drugs. Proteins with a lower MW, less than about 60-70 kD, are particularly good candidates because of the porosity of the gel-network. In some embodiments, the cells are present as multicellular aggregates or tissue.

This invention is useful in biomedical applications where a pH-neutral polysaccharide foam composition is desired e.g. cell culture matrix, tissue engineering scaffold, and implantation applications such as anti adhesion barrier. It is compatible with living cells or tissue or other pH sensitive components such as drugs and/or peptides or proteins that require neutral pH.

### EXAMPLES

Glossary
Sodium alginate PRONOVA® MVG; batch: 701-256-11, viscosity (1 wt % aqueous solution at 20° C.)=385 mPas (FMC BioPolymer, Philadelphia Pa.)
Sorbitol Sorbitol special; SPI Polyols, New Castle, USA
Glycerine Glycerol, Ph. Eur; VWR Prolabo, Leuven, Belgium
HMPC Hypromellose USP; Substitution type 2910, Pharmacoat 603, Shin-Etsu Chemical Co. Ltd., Japan

### Example 1

33% saturated foam prepared using 100% saturated Sr-alginate particles (FP-502-02, particle size <75 μm, M content about 53%.)

100 g of a 4% alginate solution made from MQ-water and sodium alginate was added to a mixing bowl. Then 18.0 g sorbitol special, 6.0 g glycerine, 3.0 g HPMC and 51.0 g MQ-water were added to the same mixing bowl. The ingredients were blended with use of a wire whisk and a Hobart mixer at medium speed for two minutes to ensure homogeneity. Then the mixing and aeration continued for six minutes at high speed. 2.0 g Sr-alginate (particle size <75 microns) dispersed in 20.0 g MQ-water was then added to the bowl with the foam, and mixing continued at high speed for another 20 seconds. The resulting foam had a wet density of 0.25 g/ml. The foam was immediately transferred to a 4 mm deep mold and the foam was kept uncovered at the laboratory bench for one hour to allow ion diffusion. Finally the foam was dried in an air forced drying oven at 80° C. for one hour. The amount of strontium ion added was sufficient to saturate 33% of the alginate in the foam (alginate from both Na-alginate and Sr-alginate).

The dried foam sheet was soft, flexible and granulated. While some cracking was seen, generally the dry foam was integral with no holes. The foam swelled fast when water was added, then it fast lost its integrity.

### Example 2

33% saturated foam prepared using a 100% saturated Sr-alginate particles (J74-037, 20 g Sr-alginate particles suspended in 450 ml water), dp₅₀˜1 μm after milling with use of an agitated ball mill. The M content of the Sr-alginate was about 41%.

100 g of a 4% alginate solution made from MQ-water and sodium alginate was added to a mixing bowl. Then 18.0 g sorbitol special, 6.0 g glycerine, 3.0 g HPMC and 26.0 g MQ-water were added to the same mixing bowl. The ingredients were blended with use of a wire whisk and a Hobart mixer at medium speed for two minutes to ensure homogeneity. Then the mixing and aeration continued for seven minutes at high speed. 47.0 g of the Sr-alginate dispersion was added the mixing bowl and mixing were continued at high speed for 25 seconds. The resulting foam had a wet density of 0.31 g/ml. Molding, gelling and drying were as described in Example 1. The Strontium added was sufficient to saturate 33% of the total alginate in the foam.

The Sr-alginate particles were visible in the wet alginate foam as gelled small fibers. The dried foam had a very coarse structure, and an open structure with holes through the foam was seen. The foam absorbed water and kept some integrity, but it was very weak.

### Example 3

25% saturated foam prepared using a 50% saturated Sr-alginate particles (FP-411-06, with a M content of about 46%), particle size: <0.25 μm.

100 g of a 4% alginate solution made from MQ-water and sodium alginate was added to a mixing bowl. Then 18.0 g sorbitol special, 6.0 g glycerine, 3.0 g HPMC and 39.0 g MQ-water were added to the same mixing bowl. The ingredients were blended with use of a wire whisk and a Hobart mixer at medium speed for two minutes to ensure homogeneity. Then the mixing and aeration continued for six minutes at high speed. 4.0 g Sr-alginate were suspended in 300 g MQ-water and added to the bowl. Mixing continued at high speed for 1 minute and 15 seconds. The wet foam gelled very fast and it was difficult to transfer the foam to the tray and get a smooth surface on the top. Molding, gelling and drying steps were as in Example 1. The Strontium added was sufficient to saturate 25% of the alginate.

The dried foam had collapsed a lot due to the large amount of water and it was somewhat less pliable than the other foams. The hydration rate of the dried foam was somewhat slower than for the other foams and it lost its integrity short time after hydration.

### Example 4

50% saturated foam using 100% saturated calcium alginate particles (FP-502-01, from the same source of sodium alginate as in example 1), particle size: >75 μm.

100 g of an alginate 4% solution made from MQ-water and sodium alginate was added to a mixing bowl. Then 18.0 g sorbitol special, 3.0 g HPMC and 69.0 g MQ-water were added to the same mixing bowl. The ingredients were blended with use of a wire whisk and a Hobart mixer at medium speed for two minutes to ensure homogeneity. Then the mixing and aeration continued for five minutes and 30 seconds at high speed. 4.0 g Ca-alginate was suspended in 9.0 g glycerine and 10.0 g water and added to the bowl. The suspension was further mixed for 30 seconds. The resulting foam had a wet density of 0.22 g/ml. Molding, gelling and drying are as in Example 1. The calcium added was sufficient to saturate 50% of the alginate.

The dried foam sheet was soft, flexible and granulated, but more homogeneous than the foams made in the previous examples. It swelled fast and then the weak wet foam disintegrated.

1. A method of producing a gelled foam comprising the steps of:
(a) forming a dispersion by mixing i) a solution comprising a soluble polysaccharide and a plasticizer and adding a polysaccharide/gel-forming ion particles or ii) a soluble, preferably immediately soluble, polysaccharide, preferably an alginate, a polysaccharide/gel-forming ion particles, and adding a solvent, said dispersion (ii) further comprising a water soluble plasticizer;
(b) aerating the dispersion in (a), said dispersion optionally further comprising a foaming agent;
(c) optionally dispensing the wet foam; and
(d) optionally drying the wet foam.

2. A method according to claim 1 in which the plasticizer is selected from glycerin, sorbitol, or a mixture thereof.

3. A method according to claim 1 in which the foaming agent is selected from an ionic surfactant, a non ionic surfactant, a foam stabilizing hydrocolloid.

4. A method according to claim 3 in which the foaming agent selected from hydroxylpropylmethylcellulose, methyl cellulose and albumin.

5. A method according to claim 1 in which the foaming agent is polymeric and biologically-acceptable and substantially free of a non-polymeric surfactant.

6. A method according to claim 1 in which the foaming agent is present in the aqueous dispersion at a level of about 0.5 wt % to about 5 wt %.

7. A method according to claim 1 in which the gel-forming ion in the polysaccharide/gel-forming ion particles comprises calcium ion, strontium ion, barium ion or mixtures thereof.

8. A method according to claim 7 in which the ion is calcium ion.

9. A method according to claim 1 in which the soluble polysaccharide and the polysaccharide in the polysaccharide/gel-forming ion are independently selected from alginates, pectins, carrageenans, chitosan, hyaluronates.

10. A method according to claim 9 in which the soluble polysaccharide is an alginate.

11. A method according to claim 10 or claim 9 in which the polysaccharide in the polysaccharide/gel-forming ion is an alginate

12. A method according to claim 1 in which the gel-forming ion from the polysaccharide/gel-forming ion particles is present at a level sufficient to saturate from about 10% to about 90% and preferably from 25 to 75% of the gelling sites of the total polysaccharide in the dispersion.

13. A method according to claim 1 in which the particle size of the polysaccharide/gel-forming ion particles is from about 500 μm to about 0.001 μm, preferably from about 100 μm to about 0.01 μm and more preferably from about 50 μm to about 0.1 μm.

14. A method according to claim 1 in which the aqueous solution in i) comprises from about 0.1 wt % to about 10 wt %, preferably 0.5 to 8% of the soluble polysaccharide.

15. A method according to claim 1 in which the dispersion comprises from about 0.5 wt % to about 10 wt %, preferably 1 to 5% of the polysaccharide/gel-forming ion particles.

16. A method according to claim 1 in which the dispersion comprises from about 2 wt % to about 25 wt %, preferably 5 to 20 wt %, more preferably 7 wt % to about 15 wt % of plasticizer.

17. A method according to claim 1 in which the soluble polysaccharide is alginate and has a G-content of greater than 50% and a molecular weight from about 10,000 Daltons to about 500,000 Daltons.

18. A method according to claim 1 in which the polysaccharide in the polysaccharide/gel-forming ion particles is alginate and has a G-content from about 30% to about 80% and a molecular weight from about 100 Daltons to about 300,000 Daltons.

19. A foam obtainable by the method according to claim 1 having an endotoxin content of less than 500 EU/gram.

20. A foam according to claim 19 having an endotoxin content of less than 100 EU/gram.

21. A foam according to claim 19 having an endotoxin content suitable for implantation into living organisms.

22. A foam according to claim 19 further comprising one or more cell growth promoting substance.

23. A foam according to claim 19 further comprising one or more cell growth inhibiting substance.

24. A foam according to claim 19 further comprising hydroxyapatite, tricalcium phosphate, demineralized bone and/or organic bone components, bone morphogenic protein, or both.

25. A foam according to claim 19 in which the soluble polysaccharide from the solution and the polysaccharide of the particle are non-uniformly distributed through the foam.

26. A foam according to claim 19 having a inhomogeneous structure.

27. An in vitro cell culture matrix or an in vivo tissue engineering scaffold comprising a self-gelling foam according to claim 19 and cells.

28. A topical wound healing bandage, a structure for treatment of burns or an anti-adhesion barrier comprising a foam according to claim 19.

29. A pharmaceutical delivery device comprising a foam according to claim 19 and a pharmaceutical to be delivered.

30. A method of pharmaceutical delivery comprising applying topically to an external or internal membrane or implanting a structure comprising the pharmaceutical to be delivered and a foam according to claim 19 and optionally dissolving the said foam an aqueous solution of citrate, EDTA or hexametaphosphate or other chelating agents for divalent ions.
